(19) United States
(12) Patent Application Publication
(10) Pub. No.: US 2020/0367849 A1
(43) Pub. Date: Nov. 26, 2020

(54) IMAGE FUSION-GUIDANCE DEVICE AND METHOD BASED ON RESPIRATORY GATING AND COMPUTED TOMOGRAPHY

(71) Applicants: HEFEI CAS ION MEDICAL AND TECHNICAL DEVICES CO., LTD, Hefei (CN); INSTITUTE OF PLASMA PHYSICS CHINESE ACADEMY OF SCIENCES, Hefei (CN)

(72) Inventors: Hansheng Feng, Hefei (CN); Shi Li, Hefei (CN); Zhu Li, Hefei (CN); Hailin Cao, Hefei (CN); Liping Liu, Hefei (CN); Yanxin Zhu, Hefei (CN); Jing Zhang, Hefei (CN); Jiajin Fu, Hefei (CN); Xinjun Zhu, Hefei (CN); Kaizhong Ding, Hefei (CN); Gen Chen, Hefei (CN); Yonghua Chen, Hefei (CN); Yuntao Song, Hefei (CN)

(21) Appl. No.: 16/992,302

(22) Filed: Aug. 13, 2020

Related U.S. Application Data
(63) Continuation of application No. PCT/CN2018/119437, filed on Dec. 6, 2018.

(30) Foreign Application Priority Data
Nov. 15, 2018 (CN) 201811359365.8

(51) Int. Cl. A61B 6/00; G06T 3/00; G06T 7/33; A61B 6/03

(52) U.S. Cl. CPC A61B 6/541; G06T 3/0068; G06T 7/33; A61B 6/035; G06T 2207/20021; A61B 6/5235; G06T 2207/10081; G06T 2207/20221; A61B 6/4435

(57) ABSTRACT

An image fusion-guidance device based on respiratory gating and CT includes a CT scanner, which is provided with a respiratory gating device and a gantry. A patient couch and a treatment head are provided in the gantry. A respiratory gating sensor is provided on the patient couch. The respiratory gating device is connected to a CT image workstation and an image fusion acquisition device via synchronization signal lines. The image fusion acquisition device is connected to an image fusion-processing device, which is connected to an image fusion display device. The treatment head is connected to a beam output controller via a beam output control line. The present application further provides an image fusion-guidance method based on respiratory gating and CT. In the guiding process, the patient is only required to hold the breath once, and then the patient breathes freely in the subsequent treatment.

### CROSS-REFERENCE TO RELATED APPLICATIONS

This application is a continuation of International Patent Application No. PCT/CN2018/119437, filed on Dec. 6, 2018, which claims the benefit of priority from Chinese Patent Application No. 201811359365.8, filed on Nov. 15, 2018. The content of the aforementioned applications, including any intervening amendments thereto, is incorporated herein by reference.

### TECHNICAL FIELD

The present application relates to medical equipment, and more particularly to an image fusion-guidance device and method based on respiratory gating and computed tomography (CT).

### BACKGROUND

Currently, the development of medical electronic equipment has made a great contribution to the development of advanced treatment techniques. However, the existing medical electronic equipment applied in some treatment methods still needs to be improved. For example, during radiation therapy, the patient is generally immobilized at an appropriate position of the patient couch, and then the patient couch is moved to a designated position. In practice, it is hard for the technicians to know the precise position of the patient relative to the rotating gantry, the treatment head, etc. In the current radiotherapy process, the breathing of the patient causes the movement of tumor and important organs, which leads to low-dose radiation on the tumor of interest or high-dose radiation on important organs that is undesired, causing radiation-induced pneumonia, hepatitis, etc. Respiratory movement significantly affects the tumor location of the chest and upper abdomen, because lesions generally occupy a small area of the patient body, and the respiratory movement of the patient leads to large tumor displacements, which increases the risk of tumor targeting failure. Thus, the respiratory gating technique is adopted in the irradiation of the localized lesions, which compensates the effect of the respiratory movement on the lesion locations and further ensures the accuracy of radiotherapy, thereby reducing the radiation complications. In addition, breath training is required before the use of the existing respiratory gating technique, which is a high requirement for patients, especially for those physically weak or old patients. In the present application, the gating technique is introduced to locate the tumor area of interest. In the clinical process, the patients that require radiotherapy, especially the patients in advanced stages (stage III, stage IV), are relatively physically weak, and it is difficult for these patients to complete the acquisition of accurate gate control signals. Moreover, during the treatment process, the patients are required to hold the breath for a long time, which is unfavorable for the clinical treatment.

In view of the above problems, an image fusion-guidance device and method based on respiratory gating and CT are provided herein to accurately identify the tumor locations while the patient breaths freely and ensure an appropriate radiation dose to be delivered to the tumor target areas. During the radiotherapy, the patient is only required to hold the breath once and then breathes freely in the subsequent process, benefiting the clinical treatment of the patient.

### SUMMARY

The present application aims to provide an image fusion-guidance device and method based on respiratory gating and CT. In the guiding process, a patient is only required to hold the breath once after a respiratory gating device is switched on, and then the patient breathes freely in the subsequent treatment. In the guiding process, more accurate guidance positioning and higher guidance efficiency are realized by a registration fusion algorithm, thereby reducing the treatment time and radiation time of the patient.

The technical solutions of the present application are described as follows.

In one aspect, the present application provides an image fusion-guidance device based on respiratory gating and computed tomography (CT), comprising: a CT scanner which is provided with a respiratory gating device and a gantry;

wherein a patient couch and a treatment head are provided in the gantry; a respiratory gating sensor is provided on the patient couch; the respiratory gating device is connected to a CT image workstation and an image fusion acquisition device via synchronization signal lines; the image fusion acquisition device is connected to an image fusion-processing device, which is connected to an image fusion display device; and the treatment head is connected to a beam output controller via a beam output control line;

the respiratory gating sensor is configured to perform real-time acquisition of free-breathing CT images which are dynamic and breath-hold CT images which are static and send the acquired CT images to the CT image workstation via synchronization signal lines; CT image workstation sends the acquired dynamic CT images and static CT images to the image fusion acquisition device; and the image fusion acquisition device sends the acquired CT images to the image fusion-processing device.

In another aspect, the present application provides an image fusion-guidance method based on respiratory gating and CT using the image fusion-guidance device, comprising:

1) switching on the respiratory gating device; acquiring signals from the respiratory gating sensor; and starting signal transmission of synchronization signal lines, which are connected to the respiratory gating device, the image fusion acquisition device, the image fusion-processing device and the image fusion display device, respectively;

2) acquiring the breath-hold CT images that are static by the CT image workstation; and transmitting the acquired static CT images to the image fusion-processing device through the image fusion acquisition device;

3) turning off the respiratory gating device; and acquiring, by the image fusion acquisition device, the free-breathing CT images that are dynamic and are sent from the CT image workstation in real time; and

4) performing registration and fusion of the dynamic CT images and the static CT images by the image fusion-processing device; when the registered and fused images reach a pre-determined threshold, outputting signals to the beam output controller; sending, by the beam output controller, beam output control signals to the treatment head via beam output control lines; and controlling the treatment head to move for therapy.

In an embodiment, performing registration and fusion of the dynamic CT images and the static CT images by the image fusion-processing device comprising:

1) under the conditions that the respiratory gating device is switched on and the patient holds the breath, acquiring, by the CT image workstation, the CT images stored or background image sequences acquired in real time to establish a statistical model for each pixel;

2) setting P and Q as point cloud data sets to be registered; finding out a corresponding point pᵢ in P that is closest to a point qᵢ in Q; wherein a relationship between a rotation matrix R and translation vectors T, P, Q is expressed as:

Qᵢ=R·Pᵢ+T(i=1, . . . ,N) (1);

establishing a registration model based on a minimum criterion of the sum of Euclidean distance between corresponding points, as shown in Function (2):

$${Σ}^{2} = {\sum }_{i = 1}^{N} P {\hat{Q}}_{i} - ( Rg {\hat{P}}_{i} + T ) {P}^{2} = min , \qquad ( 2 )$$

wherein \hat{p}ᵢ is an estimate value of Pᵢ and \hat{Q}ᵢ is an estimate value of Qᵢ;

3) centralizing the point sets Pᵢ and Qᵢ by a singular value decomposition method to prevent individual coordinates from deviating from an origin point, so as to avoid severe condition of results; wherein Pi and Qi are respectively expressed in Equation (3):

$$p = \frac{1}{N} {\sum }_{i = 1}^{N} {P}_{i} , q = \frac{1}{N} {\sum }_{i = 1}^{N} {Q}_{i} ; \qquad ( 3 )$$

carrying out rigid transformation based on Equations (1) and (3) to obtain a rigid transformation vector f(R) that minimizes a function value of Function (2), as shown in Equation (4):

$$f ( R ) = {\sum }_{i = 1}^{N} {Pq}_{i} - {Rgp}_{i} {P}^{2} = {\sum }_{i = 1}^{N} ( {q}_{i}^{T} {q}_{i} + {p}_{i}^{T} {p}_{i} - 2 {q}_{i}^{T} {Rgp}_{i} ) , \qquad ( 4 )$$

wherein Pᵢ=Pᵢ−p; Qᵢ=Qᵢ−p;

minimizing f(R) by constructing a matrix

$$H = {\sum }_{i = 1}^{N} {p}_{i} {q}_{i}^{T}$$

followed by performing a singular value decomposition to obtain orthogonal matrices U and V and a non-negative diagonal matrix λ, thus obtaining a rotation matrix R and a translation matrix T, which are expressed as

R=VU^{T},T=q−R·p (5);

4) dividing space points into a plurality of groups according to a simplex method, wherein each group consists of four nearest neighbor points; finding a closest point based on their simplicity and refining one by one; and sorting and dividing the space points by a Delaunay triangulation, thereby significantly improving the computational efficiency of the fusion registration algorithm.

The present application has the following beneficial effects.

In the guiding process, the patient is only required to hold the breath once after the respiratory gating device is switched on, and then the patient breathes freely in the subsequent treatment. Moreover, in the guiding process, more accurate guidance positioning and higher guidance efficiency are realized by a registration fusion algorithm, thereby reducing the treatment time and radiation time of the patient.

### BRIEF DESCRIPTION OF THE DRAWINGS

The present application is further described below with reference to the accompanying drawings, so that the present invention is more understandable for those skilled in the art.

FIG. 1 is a schematic diagram of an image fusion-guidance device based on respiratory gating and CT according to the present application.

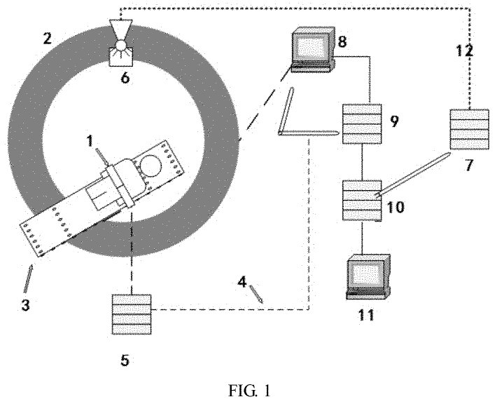

FIG. 2 is a flowchart showing a fusion registration algorithm according to the present application.

### DETAILED DESCRIPTION OF EMBODIMENTS

The present application will be described in detail below with reference to the embodiments and the accompanying drawings.

The present application provides an image fusion-guidance device based on respiratory gating and CT, including: a CT scanner. The CT scanner is provided with a respiratory gating device 5 and a gantry 2.

A patient couch 3 and a treatment head 6 are provided in the gantry 2. A respiratory gating sensor 1 is provided on the patient couch 3. The respiratory gating device 5 is connected to a CT image workstation 8 and an image fusion acquisition device 9 via synchronization signal lines 4. The image fusion acquisition device 9 is connected to an image fusion-processing device 10, which is connected to an image fusion display device 11. The treatment head 6 is connected to a beam output controller 7 via a beam output control line 12.

The respiratory gating sensor 1 is configured to perform real-time acquisition of free-breathing CT images that are dynamic and breath-hold CT images that are static, and send the acquired CT images to the CT image workstation via synchronization signal lines 4. The CT image workstation 8 sends the acquired dynamic CT images and static CT images to the image fusion acquisition device 9. The image fusion acquisition device 9 sends the acquired CT images to the image fusion-processing device 10.

The present application further provides an image fusion-guidance method based on respiratory gating and CT, which is implemented as follows.

1) The respiratory gating device 5 is switched on. Signals from the respiratory gating sensor 1 are acquired. Signal transmission of synchronization signal lines 4 which are connected to the respiratory gating device 5, the image fusion acquisition device 9, the image fusion-processing device 10 and the image fusion display device 11, respectively, is started.

2) Under the condition that the patient holds the breath, the breath-hold CT images that are static are acquired by the CT image workstation 8. The acquired static CT images are transmitted to the image fusion-processing device 10 through the image fusion acquisition device 9.

3) The respiratory gating device is turned off. Under the condition that the patient keeps breathing freely, the image fusion acquisition device 9 acquires the dynamic CT images that are transmitted from the CT image workstation in real time.

4) Registration and fusion of the dynamic CT images and the static CT images are performed by the image fusion-processing device 10. When the registered and fused images reach a pre-determined threshold, signals are output to the beam output controller 7. Beam output control signals are sent to the treatment head 6 via beam output control lines 12 by the beam output controller 7. The treatment head 6 is controlled to move for therapy.

In an embodiment, registration and fusion of the dynamic CT images and the static CT images by the image fusion-processing device are performed as follows.

1) Under the conditions that the respiratory gating device is switched on and the patient holds the breath, the CT images stored or background image sequences acquired in real time are acquired by the CT image workstation 8 to establish a statistical model for each pixel.

2) P and Q are set as point cloud data sets to be registered, that is, P and Q are respectively a regional set of images for a certain area. A corresponding point pᵢ in P that is closest to a point qᵢ in Q is found out. There is a relationship between a rotation matrix R and translation vectors T, P, Q, which is expressed as follows:

Qᵢ=R·Pᵢ+T(i=1, . . . ,N) (1).

A registration model based on a minimum criterion of the sum of Euclidean distance between corresponding points is established, as shown in Function (2):

$${Σ}^{2} = {\sum }_{i = 1}^{N} P {\hat{Q}}_{i} - ( Rg {\hat{P}}_{i} + T ) {P}^{2} = min , {d}_{N - 1} = \sqrt{{Σ}^{2}} , \qquad ( 2 )$$

where \hat{p}ᵢ is an estimate value of Pᵢ and \hat{Q}ᵢ is an estimate value of Qᵢ.

3) The point sets Pᵢ and Qᵢ are centralized by a singular value decomposition method to prevent individual coordinates from deviating from an origin point, so as to avoid severe condition of results. Pi and Qi are respectively expressed in Equation (3):

$$p = \frac{1}{N} {\sum }_{i = 1}^{N} {P}_{i} , q = \frac{1}{N} {\sum }_{i = 1}^{N} {Q}_{i} . \qquad ( 3 )$$

Rigid transformation is carried out based on Equations (1) and (3) to obtain a rigid transformation vector f(R) that minimizes a function value of Function (2), as shown in Equation (4):

$$f ( R ) = {\sum }_{i = 1}^{N} {Pq}_{i} - {Rgp}_{i} {P}^{2} = {\sum }_{i = 1}^{N} ( {q}_{i}^{T} {q}_{i} + {p}_{i}^{T} {p}_{i} - 2 {q}_{i}^{T} {Rgp}_{i} ) , τ = \frac{\sqrt{f ( R )}}{N} , \qquad ( 4 )$$

where Pᵢ=Pᵢ−p; and Qᵢ=Qᵢ−p.

In order to minimize f(R), a matrix

$$H = {\sum }_{i = 1}^{N} {p}_{i} {q}_{i}^{T}$$

can be constructed followed by performing a singular value decomposition to obtain orthogonal matrices U and V and a non-negative diagonal matrix λ, and thus, a rotation matrix R and a translation matrix T are obtained and expressed as

R=VU^{T},T=q−R·p (5).

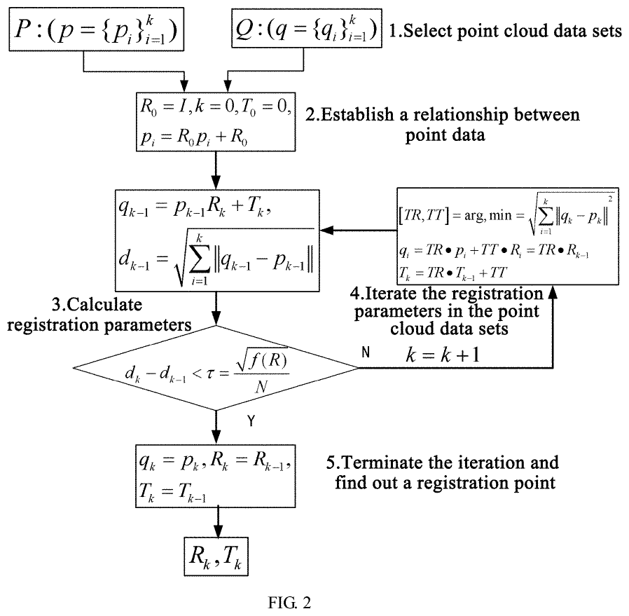

4) Space points are divided into a plurality of groups according to a simplex method, and each group consists of four nearest neighbor points. A closest point is found out based on their simplicity and by refining one by one. The space points are sorted and divided using a Delaunay triangulation, thereby significantly improving the computational efficiency of the fusion registration algorithm. FIG. 2 shows a flowchart of the fusion registration algorithm, which is specified as follows.

1) Corresponding point sets are triangulated. Parameters are initialized according to R=E_{3×3}, T=[0 0 0]^{T}.

2) A closest point set Qₖ is calculated as a corresponding point of Pₖ according to Qₖ=RPₖ+T.

3) Registration parameters TR, TT, dₖ are calculated.

4) The registration parameters are plugged into a target point set P₀ to obtain a new coordinate Pₖ₊₁=RₖPₖ₋₁+Tₖ.

5) If the change of dₖ is less than a given threshold T, the iteration is terminated. Otherwise, steps (2)-(5) are repeated.

Described above are merely preferred embodiments, which are illustrative without the detailed descriptions, and are not intended to limit the scope of the present application. Obviously, many modifications and changes can be made according to the present description. The embodiments in the present application are specifically described to better explain the principles and practical applications, so that the present application is more understandable for those skilled in the art. The scope of the present application is only defined by the appended claims and the equivalents.

### INDUSTRIAL APPLICABILITY

In the guiding process, the patient is only required to hold the breath once after the respiratory gating device is switched on, and then the patient breathes freely in the subsequent process. Moreover, in the guiding process, more accurate guidance positioning and higher guidance efficiency are realized by a registration fusion algorithm, thereby reducing the treatment time and radiation time of the patients.

What is claimed is:

1. An image fusion-guidance device based on respiratory gating and computed tomography (CT), comprising: a CT scanner, which is provided with a respiratory gating device and a gantry;
Where in a patient couch and a treatment head are provided in the gantry; a respiratory gating sensor is provided on the patient couch; the respiratory gating device is connected to a CT image workstation and an image fusion acquisition device via synchronization signal lines; the image fusion acquisition device is connected to an image fusion-processing device, which is connected to an image fusion display device; and the treatment head is connected to a beam output controller via a beam output control line; and
the respiratory gating sensor is configured to perform real-time acquisition of free-breathing CT images that are dynamic and breath-hold CT images that are static, and send the acquired CT images to the CT image workstation via synchronization signal lines; CT image workstation sends the acquired dynamic CT images and static CT images to the image fusion acquisition device; and the image fusion acquisition device sends the acquired CT images to the image fusion-processing device.

2. An image fusion-guidance method based on respiratory gating and CT using the image fusion-guidance device of claim 1, comprising:
1) switching on the respiratory gating device; acquiring signals from the respiratory gating sensor; and starting signal transmission of synchronization signal lines which are connected to the respiratory gating device, the image fusion acquisition device, the image fusion-processing device and the image fusion display device, respectively;
2) acquiring the breath-hold CT images that are static by the CT image workstation; and transmitting the acquired static CT images to the image fusion-processing device through the image fusion acquisition device;
3) turning off the respiratory gating device; and acquiring, by the image fusion acquisition device, the free-breathing CT images that are dynamic and are sent from the CT image workstation in real time; and
4) performing registration and fusion of the dynamic CT images and the static CT images by the image fusion-processing device; when the registered and fused images reach a pre-determined threshold, outputting signals to the beam output controller; sending, by the beam output controller, beam output control signals to the treatment head via beam output control lines; and controlling the treatment head to move for therapy.

3. The image fusion-guidance method of claim 2, wherein performing registration and fusion of the dynamic CT images and the static CT images by the image fusion-processing device comprising:
1) under the condition that the respiratory gating device is switched on and the patient holds the breath, acquiring, by the CT image workstation, the CT images stored or background image sequences acquired in real time to establish a statistical model for each pixel;
2) setting P and Q as point cloud data sets to be registered; finding out a corresponding point pᵢ in P that is closest to a point qᵢ in Q; wherein a relationship between a rotation matrix R and translation vectors T, P, Q is expressed as:
Qᵢ=R·Pᵢ+T(i=1, . . . ,N) (1);
establishing a registration model based on a minimum criterion of the sum of Euclidean distance between corresponding points, as shown in Function (2):
$${Σ}^{2} = {\sum }_{i = 1}^{N} P {\hat{Q}}_{i} - ( Rg {\hat{P}}_{i} + T ) {P}^{2} = min , \qquad ( 2 )$$
wherein \hat{p}ᵢ is an estimate value of Pᵢ and \hat{Q}ᵢ is an estimate value of Qᵢ;
3) centralizing the point sets Pᵢ and Qᵢ by a singular value decomposition method to prevent individual coordinates from deviating from an origin point, so as to avoid severe condition of results; wherein Pᵢ and Qᵢ are respectively expressed in Equation (3):
$$p = \frac{1}{N} {\sum }_{i = 1}^{N} {P}_{i} , q = \frac{1}{N} {\sum }_{i = 1}^{N} {Q}_{i} ; \qquad ( 3 )$$
carrying out rigid transformation based on Equations (1) and (3) to obtain a rigid transformation vector f(R) that minimizes a function value of Function (2), as shown in Equation (4):
$$f ( R ) = {\sum }_{i = 1}^{N} {Pq}_{i} - {Rgp}_{i} {P}^{2} = {\sum }_{i = 1}^{N} ( {q}_{i}^{T} {q}_{i} + {p}_{i}^{T} {p}_{i} - 2 {q}_{i}^{T} {Rgp}_{i} ) , \qquad ( 4 )$$
wherein Pᵢ=Pᵢ−p; and Qᵢ=Qᵢ−p;
minimizing f(R) by constructing a matrix
$$H = {\sum }_{i = 1}^{N} {p}_{i} {q}_{i}^{T}$$
and performing a singular value decomposition to obtain orthogonal matrices U and V and a non-negative diagonal matrix λ, thus obtaining a rotation matrix R and a translation matrix T, which are expressed as
R=VU^{T},T=q−R·p (5); and
4) dividing space points into a plurality of groups according to a simplex method, wherein each group consists of four nearest neighbor points; finding out a closest point based on their simplicity and refining one by one; and sorting and dividing the space points by a Delaunay triangulation.
